(19) United States
(12) Patent Application Publication
(10) Pub. No.: US 2025/0341531 A1
(43) Pub. Date: Nov. 6, 2025

(54) DETERMINING AN AMOUNT OF ANALYTE IN PLASMA BASED ON A MEASUREMENT OF AN AMOUNT OF ANALYTE IN A WHOLE-BLOOD SAMPLE

(71) Applicant: Radiometer Medical ApS, Brønshøj (DK)

(72) Inventors: Birgitte KAAE, Brønshøj (DK); John Michael PETERSEN, Brønshøj (DK); Carl Peder TROLDBORG, Brønshøj (DK); Niko PORJO, Brønshøj (DK); Henrik FODGAARD, Brønshøj (DK); Susann Irene Johanna ERIKSSON, Brønshøj (DK)

(21) Appl. No.: 18/864,305

(22) PCT Filed: May 9, 2023
(86) PCT No.: PCT/EP2023/062190

§ 371 Date: Nov. 8, 2024

(30) Foreign Application Priority Data
May 10, 2022 (EP) 22172524.5

(51) Int. Cl. G01N 33/80; G01N 33/68; G01N 33/96

(52) U.S. Cl. CPC G01N 33/80; G01N 33/6887; G01N 33/96; G01N 2333/4712; G01N 2496/05

(57) ABSTRACT

Disclosed herein are embodiments of a method for calibrating a group of analyzer units, each analyzer unit of the group of analyzer units configured for determining an amount of an analyte in plasma of a whole-blood sample. The method comprises: providing a plurality of calibration whole-blood samples, the plurality of calibration whole-blood samples including calibration whole-blood samples having respective hematocrit levels, for each calibration whole-blood sample of the plurality of calibration whole-blood samples: measuring a hematocrit measurement value indicative of the hematocrit level of said calibration whole-blood sample, measuring a whole-blood measurement value indicative of an amount of the analyte in the calibration whole-blood sample using at least one calibration analyzer unit of said group of analyzer units, measuring a plasma measurement value indicative of an amount of the analyte in plasma of said calibration whole-blood sample, and computing a ratio between the whole-blood measurement value and the plasma measurement value; generating a nonlinear functional relationship between the computed ratios and the corresponding hematocrit measurement values by curve fitting of a nonlinear function parametrized by one or more calibration parameters, the curve fitting resulting in respective parameter values of the one or more calibration parameters; storing a representation of the fitted nonlinear function in each analyzer unit of the group of analyzer units to allow each analyzer unit of the group of analyzer units to compute a hematocrit correction factor.

### TECHNICAL FIELD

The present invention relates to various aspects of determining an amount of analyte in plasma based on a measurement of an amount of analyte in a whole-blood sample.

### BACKGROUND

Analyzer units for measuring amounts of analytes in blood samples by means of respective detectors or sensors are widely used in the medical and clinical field. Such analyzer units are often simply referred to as analyzers.

In addition to general requirements in respect of accuracy, precision, and reliability, analyzer units for clinical applications are often subject to further critical constraints. Such constraints include a need for low operating costs, low down times, ease of use, efficiency of use, in particular reduced need for sample preparation, etc.

Many analyzer units perform measurements on biological samples that comprise blood with all its components, in particular including the plasma as well as the red blood cells. This has the advantage that the need for pre-processing the blood sample prior to performing the desired measurements is kept to a minimum, if not eliminated altogether. Generally, a blood sample including both plasma and red blood cells is referred to as a “whole blood” sample.

Usually, the concentration of at least some analytes is defined as the concentration of the analyte in plasma. A direct measurement of the concentration of the analyte in plasma based on a whole-blood sample would thus initially require the separation of the whole-blood sample into plasma and other blood components, in particular red blood cells. However, this is a time-consuming process.

It is therefore desirable to determine the amount of analyte in plasma directly from a measurement of an amount of the analyte in a whole-blood sample.

To this end, U.S. Pat. No. 10,132,800 proposes a method for measuring an analyte amount in a whole-blood sample, including: measuring the hematocrit level of the whole-blood sample; measuring an analyte amount directly in the whole-blood sample; and calculating a corrected analyte amount according to relation: Dₚ=Pₐ(D_{ST}, D_{H}) where Dₚ is the corrected analyte amount, D_{ST} is the measured analyte amount, D_{H} is the measured hematocrit level, and Pₐ is a non-constant polynomial of a degree greater than or equal to 1 having as indeterminate values the measured analyte amount, D_{ST}, and the measured hematocrit level, D_{H}, and having its polynomial coefficients depending on the analyte.

However, multivariate polynomials having two independent variables require relatively many polynomial coefficients to be determined in a calibration process, thus rendering the process complex and prone to overfitting. For example, even a lowest order multivariate polynomial including only a single cross-term includes four polynomial coefficients that need to be determined by calibration.

Accordingly, it remains desirable to provide a method for measuring an analyte amount in a whole-blood sample that is accurate and robust and that requires only little calibration efforts, or that at least provides an alternative to known methods.

It is further desirable to provide a method that corrects for the deviation of measurements on whole-blood samples from corresponding measurements on plasma samples without the need for repeated calibration of the individual analyzer units by the operator of the analyzer unit. Instead, it is desirable that the applicable correction can be determined during design or manufacturing of the device and/or of the associated assays and that can be sent to each analyzer unit.

### SUMMARY

On this background, according to a first aspect, disclosed herein are embodiments of a method for calibrating a group of analyzer units, each analyzer unit of the group of analyzer units configured for determining an amount of an analyte in plasma of a whole-blood sample. Embodiments of the method comprise:

a) providing a plurality of calibration whole-blood samples, the plurality of calibration whole-blood samples including calibration whole-blood samples having respective hematocrit levels,

b) for each calibration whole-blood sample of the plurality of calibration whole-blood samples:
i) measuring a hematocrit measurement value indicative of the hematocrit level of said calibration whole-blood sample,
ii) measuring a whole-blood measurement value indicative of an amount of the analyte in the calibration whole-blood sample using at least one calibration analyzer unit of said group of analyzer units,
iii) measuring a plasma measurement value indicative of an amount of the analyte in plasma of said calibration whole-blood sample, and
iv) computing a ratio between the whole-blood measurement value and the plasma measurement value;

c) generating a nonlinear functional relationship between the computed ratios and the corresponding hematocrit measurement values by curve fitting of a nonlinear function parametrized by one or more calibration parameters, the curve fitting resulting in respective parameter values of the one or more calibration parameters;

d) storing a representation of the fitted nonlinear function in each analyzer unit of the group of analyzer units to allow each analyzer unit of the group of analyzer units to compute a hematocrit correction factor.

The representation of the fitted nonlinear function is a representation of the parametrized nonlinear function where the one or more calibration parameters have the parameter values resulting from the curve fitting. Once calibrated, each of the analyzer units of the group of analyzer units may determine an amount of the analyte in plasma of a whole-blood sample by measuring a whole-blood measurement value indicative of an amount of the analyte in a whole-blood sample and a hematocrit measurement value indicative of a hematocrit level of the whole-blood sample, by determining a hematocrit correction factor from the hematocrit measurement value and from the stored representation of the fitted nonlinear function, including the fitted parameter values, and by applying the hematocrit correction factor to the whole-blood measurement value.

In particular, according to one aspect, disclosed herein are embodiments of a method for measuring an amount of analyte in plasma of a whole-blood sample; wherein the method comprises:
measuring a whole-blood measurement value indicative of a measured amount of analyte in the whole-blood sample;
measuring a hematocrit measurement value indicative of a measured hematocrit level of the whole-blood sample;
computing a hematocrit correction factor from a stored representation of a fitted nonlinear function of the measured hematocrit level, in particular a fitted nonlinear function parametrized by one or more calibration parameters having parameter values previously determined by performing the steps of the method for calibrating a group of analyzer units disclosed above and in the following;
computing the amount of analyte in plasma by applying the computed hematocrit correction factor to the whole-blood measurement value.

Accordingly, the amount of the analyte is computed as a simple product or ratio of the measurement value indicative of the measured amount of analyte in the whole-blood sample and a hematocrit correction factor that depends on the measured hematocrit level. In particular, the hematocrit correction factor is derived from a fitted nonlinear function of an indeterminate variable representing the hematocrit level, the fitted nonlinear function being parametrized by one or more calibration parameters. The parameter values of the one or more calibration parameters in the fitted nonlinear function depend on at least the type of analyte and/or the type of assay used for measuring the analyte.

In some embodiments, the nonlinear function only has a single indeterminate variable, in particular the hematocrit level as the only indeterminate variable, thus facilitating an accurate and robust calibration fit using only a few calibration parameters. In some embodiments, the nonlinear function is further dependent on the measured amount of analyte, but preferably with only three or fewer calibration parameters. Preferably, the calibration function is a non-polynomial function, such as including an exponential function.

In some embodiments, the nonlinear function is parameterized by fewer than four calibration parameters, such as three or two calibration parameters or a single calibration parameter.

The inventors have realized that this process provides an accurate determination of the analyte amount in plasma over a large range of analyte amounts and hematocrit levels, while reducing the risk of undesirable overfitting.

Computing the hematocrit correction factor from the stored representation of the fitted nonlinear function may comprise evaluating the fitted nonlinear function at the measured hematocrit value, either by explicit computation of the function value, by look-up in a look-up table and optional interpolation, or in another suitable manner so as to obtain the function value of the fitted non-linear function at the measured hematocrit value. In particular, in some embodiments, the nonlinear function is represented by a look-up table with the hematocrit value as the key, optionally including an interpolation. Applying the computed hematocrit value to the whole-blood measurement value may comprise multiplying the whole-blood measurement value with the computed hematocrit correction factor or dividing the whole-blood measurement value by the computed hematocrit correction factor. This may depend on whether the ratios between the whole-blood and plasma measurement during calibration have been computed by dividing the whole-blood measurement value by the corresponding plasma measurement value or by dividing the plasma measurement value by the corresponding whole-blood measurement value.

Preferably, the calibration process is performed using a more than one calibration analyzer units in order to accommodate for possible variations between analyzer units of the group of analyzer units. Accordingly, in some embodiments, step b) of the method for calibrating the group of analyzer units comprises, for each of a set of calibration analyzer units of the group of analyzer units, performing the following steps for each of a set of calibration whole-blood samples of the plurality of whole blood samples:
i) measuring a hematocrit measurement value indicative of the hematocrit level of said calibration whole-blood sample,
ii) measuring a whole-blood measurement value indicative of an amount of the analyte in said calibration whole-blood sample using said calibration analyzer unit,
iii) measuring a plasma measurement value indicative of an amount of the analyte in plasma of said calibration whole-blood sample, and
iv) computing a ratio between the whole-blood measurement value and the plasma measurement value.

Preferably, the set of calibration analyzer units comprises more than one, such as more than five, such as between 3 and 20, such as between 5 and 10, e.g. between 6 and 10 calibration analyzer units. The calibration analyzer units may be used to perform measurements on the same set of calibration whole-blood sample or on respective sets of calibration whole-blood samples.

In some embodiments, during calibration, the hematocrit measurement value and/or the plasma measurement value are measured by the same calibration analyzer unit used for measuring the corresponding whole-blood measurement value of the calibration whole-blood sample. To this end, a calibration plasma sample may be prepared from each calibration whole-blood sample, e.g. in a known manner, such as by centrifugation. The prepared calibration plasma sample may then be presented to the calibration analyzer unit for measurement of the plasma measurement value indicative of the amount of analyte in the plasma sample obtained from the original whole-blood sample.

Accordingly, in some embodiments, generating the nonlinear functional relationship by curve fitting comprises: generating a plurality of data points, each data point representing a hematocrit measurement value of a calibration whole-blood sample and a corresponding computed ratio between a whole-blood measurement value measured by one of the set of calibration analyzer units on said calibration whole-blood sample and a corresponding plasma measurement value measured by the same one of the set of calibration analyzer units. The corresponding plasma measurement value is preferably measured on a plasma sample obtained from said calibration whole blood sample. The hematocrit measurement value of the calibration whole-blood sample is preferably measured using the same one of the set of calibration analyzer units as the corresponding whole-blood measurement value pertaining to the same data point. Preferably, the curve fitting is based on data points obtained using respective ones of the set of the calibration analyzer units and data points obtained using respective ones of the plurality of calibration whole-blood samples.

In various embodiments, the calibration parameters associated with a particular analyte are determined by curve fitting the parametrized nonlinear function to a generated calibration data set. The calibration data set is derived from measured analyte amounts of said particular analyte in calibration whole-blood samples and in corresponding plasma samples. The measured analyte amounts of said particular analyte in the calibration whole-blood samples are obtained by one or more calibration analyzer units of the same group as the measurement analyzer unit used for the subsequent measurements.

In some embodiments, the parametrized nonlinear function is a nonlinear, non-polynomial function of the hematocrit level. In particular, in some embodiments, the nonlinear, non-polynomial function is an exponential function of the hematocrit level. The inventors have realized that a non-polynomial functions, in particular an exponential function, provides a particular accurate correction factor with a low risk of overfitting, at least for some types of analytes. In some embodiments, the non-polynomial function is a function different from a fraction of two polynomials.

In some embodiments, the hematocrit correction factor HCF (Hct) is calculated from the measured hematocrit level Hct as

$$HCF ( Hct ) = exp ( f ( Hct ) ) ,$$

where f(Hct) is a function of at least the measured hematocrit level Hct. In some embodiments, the function f is a parametrized function, parametrized by one or more parameters. In some embodiments, the function f has Hct as its only indeterminate while, in other embodiments, the function f is dependent on one or more additional quantities, e.g. temperature and/or the concentration of the analyte to be determined.

In some embodiments, the hematocrit correction factor HCF(Hct) is calculated from the measured hematocrit level Hct as

$$HCF ( Hct ) = exp ( a \cdot {Hct}^{b} ) ,$$

with calibration parameters a and b. In some embodiments, the calibration parameter a has a parameter value between 2.0 and 2.4, such as between 2.20 and 2.21, such as a=2.204 and wherein the calibration parameter b has a parameter value between 2.2 and 2.7, such as between 2.4 and 2.5, such as between 2.45 and 2.47, such as b=2.468. In another embodiment, the calibration parameter a has a parameter value between 1.9 and 2.0, such as between 1.96 and 1.97 and wherein the calibration parameter b has a parameter value between 1.5 and 1.6, such as between 1.53 and 1.54.

In some embodiments, the hematocrit correction factor HCF (Hct) is calculated from the measured hematocrit level Hct as

$$HCF ( Hct ) = exp ( a \cdot {Hct}^{b} \cdot {conc}^{c} )$$

with calibration parameters a, b and c, and where conc designates the measured amount of analyte in the whole-blood sample or an approximation thereof. In some embodiments, the calibration parameter a has a parameter value between 0.8 and 3.0, such as between 1.5 and 2.0, such as between 1.7 and 1.9. In some embodiments, the calibration parameter b has a parameter value between 1.5 and 2.0, such as between 1.7 and 1.9. The parameter c may be selected between −0.01 and 1.5, such as between −0.01 and 0.2, or between 0.05 and 1.5. In some embodiments, the parameter c may be selected in dependence of the concentration conc. For example, the parameter c may be determined from a look-up table indexed by the concentration conc. In particular, respective values of c may be associated with different concentration ranges, or the parameter c may be determined by interpolation between parameter values obtained from a look-up table, or otherwise. Accordingly, an accurate calibration may be achieved with relatively few calibration parameters.

In some embodiments, measuring the whole-blood measurement value and/or measuring the plasma measurement value comprises using an immunoassay. The fitted nonlinear function and, hence, the hematocrit correction factor may be specific to the type of immunoassay. The immunoassay may be provided in the form of a replaceable cartridge insertable into the analyzer unit. The fitted nonlinear function and, hence, the hematocrit correction factor may thus be specific to the type of immunoassay but not specific to the particular analyzer unit, as long as the analyzer unit belongs to the group of analyzer unit for which the fitted nonlinear function applies, e.g. all analyzer units of a particular make and model.

In some embodiments the analyte is an antigen. In some embodiments, the analyte is cardiac troponin I. Accordingly, in some embodiments, the whole-blood measurement value is obtained by means of a troponin I assay, in particular a high-sensitivity troponin I assay (hsTnI). Various embodiments of the method disclosed herein provide accurate measurement of analytes, in particular of hsTnI, even when the whole-blood samples are only little diluted. Accordingly, a high sensitivity may be obtained. The inventors have found that a hematocrit correction factor for hsTnI and other analytes, such as NT-proBNP and/or others, can accurately be determined based on hematocrit alone, in particular based on a nonlinear function of only the hematocrit level as indeterminate variable, independently of the analyte concentration. For some analytes, e.g. for Procalcitonin (PCT), while a nonlinear function independently of the analyte concentration may be used, a non-polynomial, nonlinear function that also depends on the analyte concentration may be particularly suitable.

The present disclosure relates to different aspects including the methods described above and in the following, corresponding apparatus, systems, methods, and/or products, each yielding one or more of the benefits and advantages described in connection with one or more of the other aspects, and each having one or more embodiments corresponding to the embodiments described in connection with one or more of the other aspects and/or disclosed in the appended claims.

In particular, according to one aspect, disclosed herein are embodiments of measuring an amount of analyte in plasma of a whole-blood sample using a measurement analyzer unit of a group of analyzer units; wherein the method comprises:
providing a plurality of calibration whole-blood samples, the plurality of calibration whole-blood samples including calibration whole-blood samples having respective hematocrit levels,
for each calibration whole-blood sample of the plurality of calibration whole-blood samples: measuring a hematocrit measurement value indicative of the hematocrit level of said calibration whole-blood sample, measuring a calibration whole-blood measurement value indicative of an amount of the analyte in the calibration whole-blood sample using at least one calibration analyzer unit of said group of analyzer units, measuring a calibration plasma measurement value indicative of an amount of the analyte in plasma of said calibration whole-blood sample, and computing a ratio between the calibration whole-blood measurement value and the calibration plasma measurement value;
generating a nonlinear functional relationship between the computed ratios and the corresponding hematocrit measurement values by curve fitting of a nonlinear function parametrized by one or more calibration parameters, the curve fitting resulting in respective parameter values of the one or more calibration parameters;
storing a representation of the fitted nonlinear function in at least the measurement analyzer unit;
measuring, by the measurement analyzer unit, a whole-blood measurement value indicative of a measured amount of the analyte in the whole-blood sample;
measuring, by the measurement analyzer unit, a hematocrit measurement value indicative of a measured hematocrit level of the whole-blood sample;
computing a hematocrit correction factor from the stored representation of the fitted nonlinear function;
computing the amount of the analyte in plasma by applying the computed hematocrit correction factor to the measured whole-blood measurement value.

According to another aspect, disclosed herein are embodiments of a computer-implemented method of determining an amount of an analyte in plasma based on a measurement of an amount of the analyte in a whole-blood sample; wherein the method comprises:
receiving a whole-blood measurement value obtained by a measurement analyzer unit, the whole-blood measurement value being indicative of a measured amount of analyte in a whole-blood sample;
receiving a hematocrit measurement value obtained by the measurement analyzer unit, the hematocrit measurement value indicative of a measured hematocrit level of the whole-blood sample;
computing a hematocrit correction factor from a stored representation of a fitted nonlinear function of the measured hematocrit level, the fitted nonlinear function being parametrized by one or more calibration parameters, in particular one or more calibration parameters having parameter values previously determined by performing the steps of the method for calibrating a group of analyzer units disclosed above and in the following;
computing the amount of the analyte in plasma by applying the computed hematocrit correction factor to the whole-blood measurement value, in particular by multiplying or dividing the whole-blood measurement value with the computed hematocrit correction factor.

Moreover, according to yet another aspect, disclosed herein are embodiments of a data processing system configured to perform the steps of the computer-implemented method described herein. In particular, the data processing system may have stored thereon program code adapted to cause, when executed by the data processing system, the data processing system to perform the steps of the computer-implemented method described herein. The data processing system may be embodied as a single computer or other data processing unit or device, or as a distributed system including multiple computers and/or other data processing devices, e.g. a client-server system, a cloud based system, etc. The data processing system may include a data storage device for storing the computer program and/or sensor data.

In some embodiments, the data processing system is integrated into the analyzer unit, e.g. as a suitably programmed internal data processing unit of the analyzer unit. In other embodiments, the data processing system may be a remote data processing system physically separate from the analyzer unit. To this end, the remote data processing system may include a communications interface for receiving measurement values from the analyzer unit, e.g. via a suitable wired or wireless connection, e.g. directly from the analyzer unit or indirectly via one or more intermediate nodes.

According to one aspect, disclosed herein are embodiments of an analyzer unit for determining an amount of analyte in plasma of a whole-blood sample; wherein the analyzer unit comprises:
an analyte sensor for measuring a whole-blood measurement value indicative of an amount of the analyte in a whole-blood sample;
a hematocrit sensor for measuring a hematocrit measurement value indicative of a hematocrit level of the whole-blood sample;
a data processing system as described above and in the following.

The analyzer unit may be an analyzer unit of a group of analyzer unit, all calibrated by the method described herein. In particular all analyzer units of the group of analyzer units may have stored thereon a representation of the same fitted nonlinear function.

The group of analyzer units may comprise or consist of analyzer units of the same make and model or otherwise analyzer units of a selected class of analyzer units that apply the same measurement protocol for measuring the analyte amount and that can therefore apply the same calibration. Hence, the calibration can be performed as part of the design of a particular analyzer unit model and/or during design of a measurement immunoassay to be used by a particular analyzer unit model for measuring a particular type of analyte.

In this respect, it will be appreciated that the parametrized nonlinear function may be analyte-specific and/or specific to a particular type of immunoassay. However, the parametrized nonlinear function may be applicable for a plurality of analyzer units, in particular to a predetermined group of analyzer units such as analyzer units of a particular make and model.

The amount of analyte determined by various embodiments of the process disclosed herein may be an absolute amount or a relative amount, in particular an analyte concentration.

Yet another aspect disclosed herein relates to embodiments of a computer program configured to cause a data processing system to perform the acts of the computer-implemented method described above and in the following. A computer program may comprise program code means adapted to cause a data processing system to perform the acts of the computer-implemented method disclosed above and in the following when the program code means are executed on the data processing system. The computer program may be stored on a computer-readable storage medium, in particular a non-transient storage medium, or embodied as a data signal. The non-transient storage medium may comprise any suitable circuitry or device for storing data, such as a RAM, a ROM, an EPROM, EEPROM, flash memory, magnetic or optical storage device, such as a CD ROM, a DVD, a hard disk, and/or the like.

### BRIEF DESCRIPTION OF THE DRAWINGS

Preferred embodiments will be described in more detail in connection with the appended drawings, where:

FIG. 1 schematically illustrates an example of an analyzer unit.

FIG. 2 schematically illustrates a process for calibrating a group of analyzer units.

FIG. 3 schematically illustrates an example of the calibration process and of a subsequent measurement process.

FIGS. 4A-C show measured data and corresponding nonlinear fits of example calibrations using different types of assays.

FIG. 5 shows the correlation of the computed analyte concentrations and reference analyte concentrations across a range of concentrations.

### DETAILED DESCRIPTION

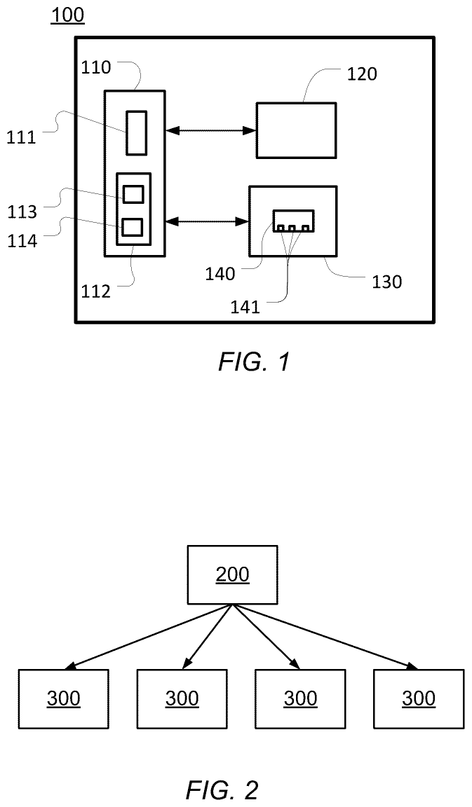

FIG. 1 schematically illustrates an example of an analyzer unit, generally designated 100. The analyzer unit comprises an analyte sensor 130, a hematocrit sensor 120, and a processing unit 110.

It will be appreciated that the analyzer unit may include one or more additional components which are not explicitly shown in FIG. 1 and which are known per se in the art of analyzer units. Examples of such additional components include a sample inlet for receiving a blood sample, a fluid handling system for presenting a received blood sample to the analyte sensor and the hematocrit sensor etc. The analyzer unit may further comprise a suitable user interface allowing a user to interact with the analyzer unit. To this end, the user interface may include a display for displaying measurement results.

The analyte sensor 130 may employ a suitable measurement methodology for measuring the concentration of one or more analytes in a whole-blood sample. In particular, the analyte sensor may be configured to measure concentration of an analyte by use of an immunoassay as is known as such in the art. To this end, the analyte sensor may be a cartridge-based immunoassay sensor and the analyzer may be configured to receive an assay cartridge 140. The cartridge may comprise a plurality of reagent cups 141.

In some embodiments, the immunoassays are based on a dry-chemistry concept and a detection method based on non-enhanced time-resolved fluorescence (TRF) technology. In this respect, the term dry-chemistry means that the required assay-specific reagents, e.g. including tracer antibody, capture antibody and stabilizing reagents, are dry-coated into one or more assay-specific reagent cups 141 of the assay cartridge 140.

In one embodiment, each reagent cup is coated with streptavidin. Biotinylated capture antibodies may be immobilized at the cup surface through the binding between streptavidin and biotin. Streptavidin and biotin form a strong non-covalent biological interaction. An insulation layer containing carbohydrates and all specific additives needed in the assay prevents any contact between capture and tracer antibodies. Europium-labeled tracer antibodies may be added on top of the insulating layer.

Generally, the cups 141 may be prepacked into sealed cartridges 140, each cartridge containing a plurality of cups, e.g. 16 cups or another suitable number of cups. The cartridge may further include a desiccant in a pouch to control humidity. Each cup may be individually sealed in a separate chamber to improve shelf life.

In some embodiments, in addition to the sample itself, the only other reagent needed to carry out an analysis is a buffer, in particular a liquid buffer, which may be the same for all tests. To this end, the analyzer unit may include an onboard solution pack, which may be a closed system containing buffer in a bag and which also has receptacles for waste collection, both waste cups and liquid waste. This means a user does not need to come into direct contact with the sample or any used reagents.

The analyte sensor is configured to analyze a blood sample. The blood sample can be either whole blood or plasma. However, during normal use of the analyzer unit, e.g. during clinical use, it is often preferred to perform the measurements directly on whole-blood samples so as to reduce the time and effort needed for sample preparation prior to the measurement. The sample may be received by the analyzer unit in a sample tube, in particular a closed sample tube. The analyzer unit may perform aspiration from the closed sample tube automatically. The analyzer unit may obtain a small amount of sample and add the obtained sample to the reagent cup. The sample is typically diluted by a buffer. When the sample (and potentially buffer) is added, it will dissolve the insulating layer of the cup. This may occur over a relatively short period of time, such as in less than 15 seconds.

The cup may be incubated at a suitable temperature, such as at 37° C. During this incubation an antibody-antigen-antibody “sandwich” complex is formed, and the complex remains immobilized at the bottom of the reagent cup by the capture antibody. The cups are washed to remove all unbound material, and dried. After drying, the analyte sensor 130 exposes the cup to excitation light and measures the europium response to the excitation light. The response may be expressed in counts per second or in another suitable manner. The response is in direct proportion to the emitted photons, which is directly proportional to the amount of antigen present. Accordingly, the measured response may serve as a measurement value indicative of the concentration of the analyte in the sample. In particular, when the sample is a whole-blood sample, the measured response may serve as a whole-blood measurement value indicative of the concentration of the analyte in the whole-blood sample. Similarly, when the sample is a plasma sample, the measured response may serve as a plasma measurement value indicative of the concentration of the analyte in the plasma sample, e.g. a plasma sample of, i.e. obtained from, a corresponding whole-blood sample.

It will be appreciated that other embodiments of an analyzer unit may include a different type of analyte sensor or be configured to perform the measurement of the analyte concentration in a different manner.

The hematocrit sensor 120 may employ a suitable measurement methodology for determining the hematocrit level of a received whole-blood sample. This may be performed in parallel with the assay measurement. Generally, the hematocrit level may be determined based on an automatic measurement of the electrical conductivity of the whole-blood sample. In one embodiment, the conductivity is measured at two frequencies. Based on the measured conductivities the Hct is determined and, optionally corrected for the salt concentration of the sample. It will be appreciated that other embodiments of an analyzer unit may include a different type of hematocrit sensor or be configured to perform the measurement of the hematocrit level in a different manner.

The analyte sensor 130 and the hematocrit sensor 120 are communicatively coupled to the processing unit 110 and forward their respective measurement results to the processing unit 110 for further processing. It will be appreciated that the sensors may forward raw measurement signals or pre-processed measurement signals or data, such as A/D converted signals, filtered signals, amplified signals and/or otherwise preprocessed signals or data to the processing unit 110.

The processing unit 110 may include a suitable programmed CPU 111 and a data storage device 112. The processing unit 110 is configured for executing program code 113 to control operation of the analyzer unit. The data storage device 112 may be a hard drive, an EEPROM, a solid-state drive, or another suitable data storage device. The data storage device may have stored thereon the program code 113. The processing unit 110 may thus load the program code 113 from the data storage device 112 into the CPU 111 which may execute the loaded program code.

In particular, the program code 113 is configured to cause the processing unit 110, responsive to the obtained measurement values from the analyte sensor and the hematocrit sensor, to process the measurement values and present the processed measurement results to the user, e.g. via a suitable display or in another form, and/or to communicate the processed measurement results to a remote data processing system. In various embodiments of the analyzer unit, the processing of the measurement values comprises the computation of an analyte concentration in plasma based on a measurement performed on a whole-blood sample as described herein. To this end, the data storage device 112 may have stored a representation 114 of a fitted nonlinear function for use by the analyzer unit for computing a hematocrit correction factor, e.g. as described in greater detail below. It will be appreciated that the representation of the fitted nonlinear function may be stored as part of the computer program or separately therefrom, e.g. as a configuration file or in another suitable manner. In some embodiments, the computer program and the representation of the nonlinear function may even be stored on different storage devices. The representation of the nonlinear function may even be accessed by the analyzer unit from a remote data storage device.

FIG. 2 schematically illustrates a process for calibrating a group of analyzer units, e.g. analyzer units of the same make and model. The calibration process 300 is performed on a set of calibration analyzer units and results in a representation of the fitted nonlinear function which represents a hematocrit-dependent correction factor for use by each analyzer unit of the group of analyzer units when performing a measurement process 200 on a whole-blood sample where the measurement result is to represent the analyte concentration in plasma. Accordingly, the representation of the fitted nonlinear function may be stored in each of the analyzer units of the group. This may be done during manufacture of the analyzer unit. Alternatively, e.g. when a new type of immunoassay is designed for use by the analyzer units, an assay-specific calibration process 300 may be performed using the set of calibration analyzer units, and the resulting representation of the new fitted nonlinear function may then be communicated to the analyzer units of the group. The representation of the fitted nonlinear function may e.g. be shipped on a suitable data carrier or downloaded onto the individual analyzer units via a suitable computer network. It will be appreciated that the representation of the nonlinear function may be specific for a particular type of immunoassay or at least for a specific type of analyte to be measured. However, for a particular assay or analyte, the representation of the nonlinear function obtained during the calibration described herein is analyzer-independent, i.e. is applicable to all analyzer units of a predetermined group, e.g. all analyzer units of the same make and model. Preferably, the obtained nonlinear function is independent of the analyte concentration. Preferably, at least for some types of analytes/assays, the obtained nonlinear function only depends on the hematocrit value. For other types of analytes/assays a non-polynomial, nonlinear function that depends on the analyte concentration may be used.

It will be appreciated that the representation of the nonlinear function may represent the nonlinear function in a number of ways, e.g. as an executable function call implementing a mathematical function, as a look-up table, optionally including an interpolation between tabulated function values, or in another suitable manner.

It will further be appreciated that the calibration analyzer units of the set of calibration analyzer units do not necessarily need to be specific analyzer units of the group of analyzer units, as the calibration may preferably be transferred from any analyzer unit of the group to another.

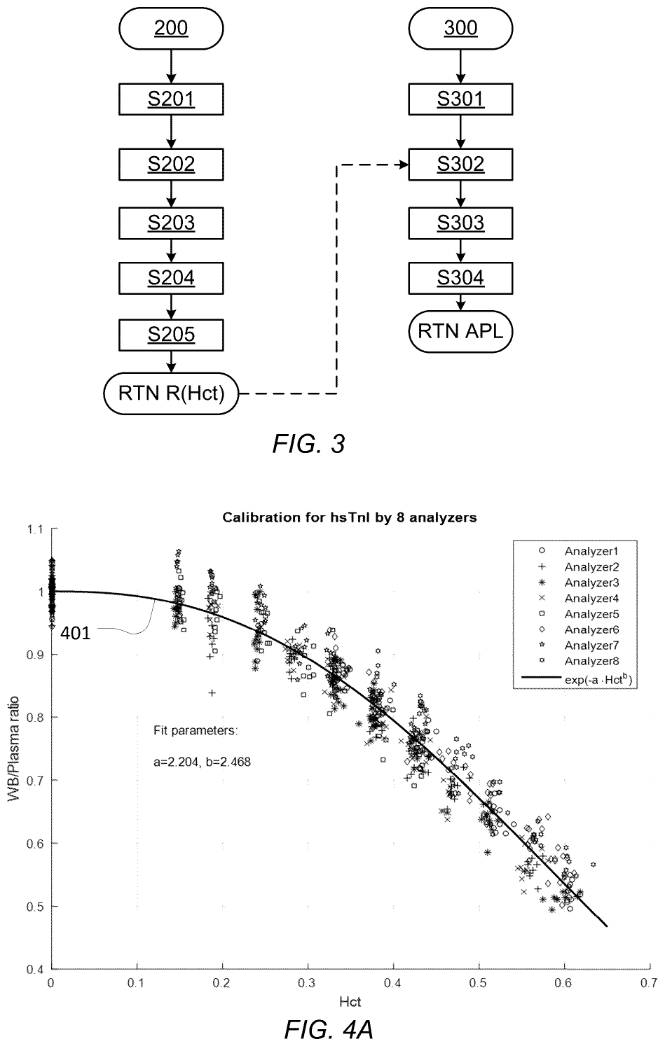

FIG. 3 schematically illustrates an example of the calibration process 300 and of a subsequent measurement process 200.

In the calibration process 300, a whole-blood-to-plasma ratio R is determined experimentally by comparing analyzer measurements from plasma samples and whole-blood samples with varying hematocrit values.

In particular, in one embodiment, in initial step S201, a plurality of calibration whole-blood (WB) samples are obtained such that the calibration whole-blood samples have hematocrit levels covering the relevant range, e.g. a range between 10 and 70% or even between 0 and 70%.

In step S202, the hematocrit level (Hct), or at least a hematocrit measurement value indicative of the hematocrit level, and a whole-blood analyte amount, or a whole-blood measurement value indicative of the whole-blood analyte amount, (AWB) are measured directly in each of the plurality of calibration whole-blood samples. The measurements are performed by a set analyzer units selected, such as randomly selected, from the group of analyzer units to be calibrated, in particular using a suitable immunoassay for measuring the analyte in question. For the purpose of the present description, the analyzer units used for performing the calibration process are also referred to as calibration analyzer units.

In step S203, the plasma analyte amount (APL), or at least a plasma measurement value indicative of the plasma analyte amount, in a plasma sample from each of the plurality of calibration whole-blood samples is measured. These measurements are preferably also performed by means of the same calibration analyzer units, using the same type of immunoassay, as the corresponding measurement of the whole-blood analyte amount.

In step S204, the process calculates the ratio R(Hct)=AWB/APL for plasma and whole-blood samples with equal analyte concentration, where AWB and APL are obtained by the same calibration analyzer unit. Respective ratios are computed for plasma and whole-blood analyte amounts measured with the calibration analyzer units of the selected set.

In step S205, the process generates a parametrized nonlinear, preferably a non-polynomial, functional relationship between the found R-values and Hct by curve fitting. The curve fitting may use any suitable fitting process, e.g. a least squares fitting process. To this end, the nonlinear functional relationship is parametrized by one or more calibration parameters and parameter values of these calibration parameters are determined by the curve fitting process, e.g. as R(Hct)=R(Hct/a₁, . . . , aₙ), with calibration parameters a₁, . . . aₙ, n>0.

In one particular embodiment, the analyte to be measured is cardiac troponin I and the assay employed for measuring the cardiac troponin I concentration in the samples is a high-sensitivity troponin I assay (hsTnI). For hsTnI the following nonlinear relationship has been found adequate:

$${R}_{h sTnI} ( H c t ) = exp ( - a \cdot {Hct}^{b} ) ,$$

with calibration parameters a and b.

In one example, the curve fitting process has resulted in a parameter value for calibration parameter a between 2.0 and 2.4, such as between 2.20 and 2.21, such as a=2.204 and in a parameter value for calibration parameter b between 2.2 and 2.7, such as between 2.4 and 2.5, such as between 2.45 and 2.47, such as b=2.468

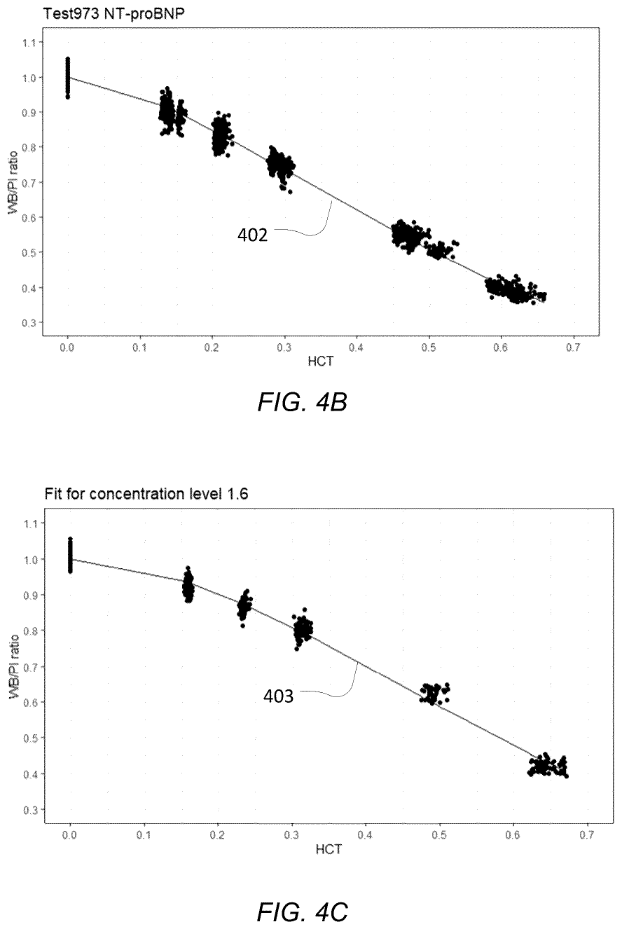

The measured data and corresponding nonlinear fit 401 of an example calibration using a hsTnI assay is illustrated in FIG. 4A. The nonlinear fit is based on data points obtained from respective calibration samples having different hematocrit levels and different analyte concentrations. Moreover, the non-linear fit is based on data points obtained using a plurality of calibration analyzer units, in this particular example eight calibration analyzer units.

For other analytes, e.g. for NT-proBNP, the above functional form may also be adequate. For example, for NT-proBNP, the following nonlinear relationship has been found suitable:

$${R}_{NT proBNP} = exp ( - a \cdot {Hct}^{b} ) ,$$

where the calibration parameter a has a parameter value between 1.9 and 2.0, such as between 1.96 and 1.97 and wherein the calibration parameter b has a parameter value between 1.5 and 1.6, such as between 1.53 and 1.54.

The measured data and corresponding nonlinear fit 402 of an example calibration using an NT-proBNP assay is illustrated in FIG. 4B. The nonlinear fit is based on data points obtained from respective calibration samples having different hematocrit levels and different analyte concentrations. Moreover, the non-linear fit is based on data points obtained using a plurality of calibration analyzer units.

The above functional relationship, using suitably selected calibration parameter values, may also be adequate for other analytes. For yet other analytes other non-linear functional relationships may also be adequate. For some analytes a functional relationship that further depends on the analyte concentration may be more suitable. For example, for PCT, the following functional relationship has been found particularly suitable, at least for a range of commonly encountered analyte concentrations:

$${R}_{PCT} = exp ( - a \cdot {Hct}^{b} * {AWB}^{c} ) ,$$

where AWB is the measured whole blood value, or an approximation thereof, e.g. a non-temperature-corrected approximation of the measured whole blood value, and with calibration parameters a, b and c. In some embodiments, the calibration parameter a has a parameter value between 0.8 and 3.0, such as between 1.5 and 2.0, such as between 1.7 and 1.9. In some embodiments, the calibration parameter b has a parameter value between 1.5 and 2.0, such as between 1.7 and 1.9. The parameter c may be selected between −0.01 and 1.5, such as between −0.01 and 0.2, or between 0.05 and 1.5. In some embodiments, the parameter c may be selected in dependence of the concentration AWB. For example, the parameter c may be determined from a look-up table indexed by the concentration AWB. In particular respective values of c may be associated to different concentration ranges, or the parameter c may be determined by interpolation between parameter values obtained from a look-up table, or otherwise.

The measured data and corresponding nonlinear fit 403 of an example calibration using a PCT assay is illustrated in FIG. 4C. The nonlinear fit is based on data points obtained from respective calibration samples having different hematocrit levels and for a given concentration range and corresponding choice of c. Moreover, the nonlinear fit is based on data points obtained using a plurality of calibration analyzer units.

Again referring to FIG. 3, in step S205, a representation, e.g. a suitable function of a computer program, or a look-up table for looking up and, optionally, interpolating function values of the nonlinear function is generated for distribution to the other analyzer units of the group of analyzer units to be calibrated.

Each of the analyzer units of the group may subsequently determine the amount of the analyte in plasma-for the analyte for which the determined nonlinear relationship is applicable-based on a measurement on a whole-blood sample. To this end, the analyzer unit used for the measurement-also referred to as the measurement analyzer unit herein-may perform the following measurement process 300:

In initial step S301 the hematocrit level Hot and the analyte amount AWB are measured directly in the whole-blood sample to be analyzed by the measurement analyzer unit.

In step S302, the hematocrit correction factor HCF(Hct) corresponding to the measured hematocrit level Hct is determined from the stored representation of the nonlinear relationship, e.g. as

$$HCF ( Hct ) = \frac{1}{R ( Hct )} .$$

In step S303, the determined hematocrit correction factor HCF(Hct) is applied to the measured analyte amount AWB to obtain the corresponding plasma analyte amount, in particular according to:

$$APL = HCF ( Hct ) \cdot AWB = \frac{AWB}{R ( Hct )} .$$

For example, for the above example of the correction factor R_{hsTnI} and with parameter values a=2.204 and b=2.468, the applicable conversion is:

$$APL = HCF \cdot AWB = AWB / {R}_{h sTnI} = AWB \cdot exp ( {Hct}^{2.468} )$$

In step S304, the process outputs, e.g. displays, the calculated plasma value APL of the analyte amount.

In the above, embodiments of a method for measuring analyte concentrations directly in whole-blood samples have been described. In various embodiments of the methods and apparatuses disclosed herein, analyte concentrations can be measured on whole-blood and plasma samples interchangeably. All reported analyte concentrations represent the analyte concentration in the plasma phase of the sample.

Thus, separating the red blood cells from the plasma and measuring on plasma, or manually determining the Hct, and manually correcting the measured analyte concentration in the whole-blood sample is not needed. The measurement is automatically conducted, and the measurements corrected and thus only the result after correction reported to the user. Moreover, the described calibration and correction have been found to be accurate and reliable, and only involve a small number of calibration parameters that need to be determined by fitting experimental data.

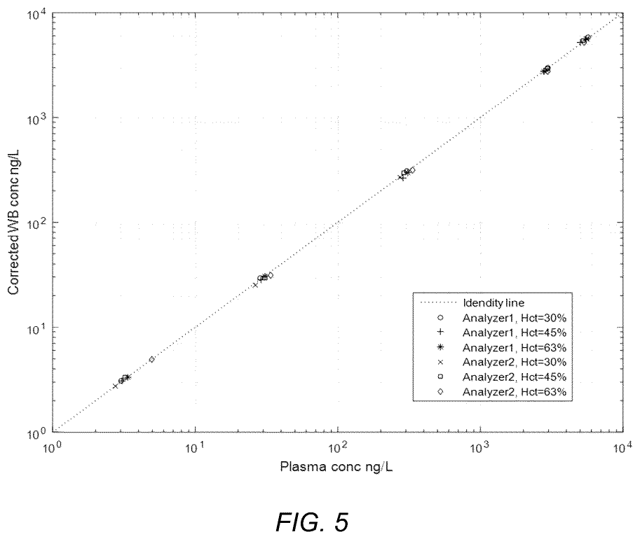

FIG. 5 shows the correlation between computed amounts of an analyte in plasma based on a measurement of an amount of the analyte in a whole-blood sample and the corresponding reference analyte concentrations in plasma across a range of concentrations. As can be seen form FIG. 5, the method described herein provides an accurate determination of the amount of analyte in plasma across a wide range of concentrations. In particular, at least for some analytes/assays, the correction factor determined as a function of only the hematocrit value has been found to be accurate independently of the analyte concentration.

Embodiments of at least some steps of the method described herein may be computer-implemented. In particular, embodiments of at least some steps of the method may be implemented by means of hardware comprising several distinct elements, and/or at least in part by means of a suitably programmed microprocessor. In the apparatus claims enumerating several means, several of these means can be embodied by one and the same element, component or item of hardware. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, elements, steps or components but does not preclude the presence or addition of one or more other features, elements, steps, components or groups thereof.

1. A method for calibrating a group of analyzer units, each analyzer unit of the group of analyzer units configured for determining an amount of an analyte in plasma of a whole-blood sample, wherein the method comprises:
providing a plurality of calibration whole-blood samples, the plurality of calibration whole-blood samples including calibration whole-blood samples having respective hematocrit levels,
for each calibration whole-blood sample of the plurality of calibration whole-blood samples: measuring a hematocrit measurement value indicative of the hematocrit level of said calibration whole-blood sample, measuring a whole-blood measurement value indicative of an amount of the analyte in the calibration whole-blood sample using at least one calibration analyzer unit of said group of analyzer units, measuring a plasma measurement value indicative of an amount of the analyte in plasma of said calibration whole-blood sample, and computing a ratio between the whole-blood measurement value and the plasma measurement value;
generating a nonlinear functional relationship between the computed ratios and the corresponding hematocrit measurement values by curve fitting of a nonlinear function parametrized by one or more calibration parameters, the curve fitting resulting in respective parameter values of the one or more calibration parameters;
storing a representation of said fitted nonlinear function in each analyzer unit of the group of analyzer units to allow each analyzer unit of the group of analyzer units to compute a hematocrit correction factor.

2. A method of measuring an amount of analyte in plasma of a whole-blood sample; wherein the method comprises:
measuring a whole-blood measurement value indicative of a measured amount of analyte in the whole-blood sample;
measuring a hematocrit measurement value indicative of a measured hematocrit level of the whole-blood sample;
computing a hematocrit correction factor from a stored representation of a fitted nonlinear function of the measured hematocrit level;
computing the amount of analyte in plasma by applying the computed hematocrit correction factor to the whole-blood measurement value.

3. A method according to claim 1; wherein the fitted nonlinear function is parameterized by fewer than four calibration parameters, such as two calibration parameters or a single calibration parameter.

4. A method according to claim 2, wherein the fitted nonlinear function is a nonlinear, non-polynomial function of the hematocrit level.

5. A method according to claim 4, wherein the nonlinear, non-polynomial function is an exponential function of the hematocrit level.

6. A method according to claim 2, wherein the analyte is an antigen.

7. A method according to claim 2, wherein the analyte is cardiac troponin I.

8. A method according to claim 7, wherein the whole-blood measurement value is obtained by means of a troponin I assay.

9. A method according to claim 2, wherein the analyte is procalcitonin or NT-proBNP.

10. A method according to claim 2, wherein the hematocrit correction factor HCF is calculated from the measured hematocrit level Hct as
$$HCF = exp ( a \cdot {Hct}^{b} ) ,$$
with calibration parameters a and b.

11. A method according to claim 10, wherein the calibration parameter a has a parameter value between 2.0 and 2.4, and wherein the calibration parameter b has a parameter value between 2.2 and 2.7.

12. A method according to claim 10, wherein the calibration parameter a has a parameter value between 1.9 and 2.0, and wherein the calibration parameter b has a parameter value between 1.5 and 1.6.

13. A method according to claim 2, wherein the hematocrit correction factor HCF is calculated from the measured hematocrit level Hct as
$$HCF ( Hct ) = exp ( a \cdot {Hct}^{b} \cdot {conc}^{c} ) ,$$
with calibration parameters a, b and c, where c is optionally dependent on the analyte concentration conc.

14. A computer-implemented method of determining an amount of an analyte in plasma based on a measurement of an amount of the analyte in a whole-blood sample; wherein the method comprises:
receiving a whole-blood measurement value obtained by a measurement analyzer unit, the whole-blood measurement value being indicative of a measured amount of analyte in a whole-blood sample;
receiving a hematocrit measurement value obtained by the measurement analyzer unit, the hematocrit measurement value indicative of a measured hematocrit level of the whole-blood sample;
computing a hematocrit correction factor from a stored representation of a fitted nonlinear function of the measured hematocrit level, the fitted nonlinear function being parametrized by one or more calibration parameters;
computing the amount of the analyte in plasma by applying the computed hematocrit correction factor to the whole-blood measurement value.

15. A computer program comprising program code configured to cause, when executed by a data processing system, the data processing system to perform the steps of the method according to claim 14.

16. A data processing system configured to perform the steps of the method according to claim 14.

17. An analyzer unit for determining an amount of analyte in plasma of a whole-blood sample; wherein the analyzer unit comprises:
an analyte sensor for measuring a whole-blood measurement value indicative of an amount of the analyte in a whole-blood sample;
a hematocrit sensor for measuring a hematocrit measurement value indicative of a hematocrit level of the whole-blood sample;
a data processing system according to claim 16.

18. An analyzer unit according to claim 17, further comprising a memory having stored thereon a representation of the fitted nonlinear function.
